(19) United States
(12) Patent Application Publication
(10) Pub. No.: US 2003/0228695 A1
(43) Pub. Date: Dec. 11, 2003

(54) Cell-manipulating apparatus and method of using the same

(75) Inventors: Chikashi Nakamura, Tokyo-to (JP); Jun Miyake, Hyogo-ken (JP); Seiji Takeda, Tokyo-to (JP); Hiroshi Tokumoto, Ibaraki-ken (JP); Masami Kageshima, Ibaraki-ken (JP); Ikuo Obataya, Tokyo-to (JP)

Correspondence Address: SUGHRUE MION, PLLC, 2100 Pennsylvania Avenue, NW, Washington, DC, 20037-3213, US

(73) Assignee: NATIONAL INSTITUTE OF ADVANCED INDUSTRIAL SCIENCE AND TECHNOLOGY

(21) Appl. No.: 10/379,701

(22) Filed: Mar. 6, 2003

(30) Foreign Application Priority Data
Mar. 6, 2002 (JP) 2002-60680
Feb. 28, 2003 (JP) 2003-54269

(51) Int. Cl. C12M 3/00; C12N 15/87

(52) U.S. Cl. 435/455; 435/470; 435/285.1

(57) ABSTRACT

The present invention provides a cell-manipulating apparatus and method, wherein a gene or a gene expression-related substance is immobilized onto a needle-shaped body such as a carbon nanotube, and the needle-shaped body is inserted into and held in a cell. The present invention allows temporal dynamic changes appearing in the cell to be intimately observed in real time from gene introduction to gene expression. The present invention is applicable to analysis or control of cell differentiation with minimized cell damage during a process of introducing the gene or the gene expression-related substance into the cell

### FIELD OF THE INVENTION

The present invention relates to a cell-manipulating apparatus and method for introducing a gene or a gene expression-related substance into a cell. In particular, the present invention relates to a cell-manipulating apparatus and method capable of allowing dynamic changes in a cell caused by a gene or a gene expression-related substance introduced thereinto to be observed in real time. The present invention is also useful for a cell differentiation control.

### BACKGROUND OF THE INVENTION

Heretofore, there have been known various conventional techniques for introducing a gene into a cell and expressing the gene, such as a physical technique including an electroporation method, a particle gun method, and a microinjection method; a technique utilizing cell-endocytosis including a calcium phosphate method, a DEAE-dextran method, and a lipofection method; and another technique including an infection method using virus vectors, and a liposome method of introducing a liposome-encapsulated gene into a cell through cell fusion. All of these gene-introducing techniques focus only on allowing the introduced gene to be permanently held in the cell but not on allowing temporal dynamic changes appearing in the cell to be observed in detail, for example, from the gene introduction to gene expression.

Actually, it has been impossible to achieve such an observation through the conventional techniques.

While temporal changes and the effects of signal transduction on expression of genes are critical factors, particularly in a cell generation/differentiation mechanism, it is difficult to analyze or control cell differentiation through the conventional techniques in which an introduced gene is permanently held in a cell. In some cases, the conventional techniques can undesirably damage a cell during the process of introducing a gene into the cell, resulting in deteriorated cell viability and increased adverse affect on subsequent experimental tests. Even if the damaged cell is repaired after the gene introduction, the time required for desirable reparation is long and unclear, and thereby the cell differentiation cannot be analyzed with a high time-resolution.

### SUMMARY OF THE INVENTION

In view of the above problems of the conventional techniques, it is therefore an object of the present invention to provide a cell-manipulating technique capable of allowing temporal dynamic changes appearing in a cell to be observed in detail, specifically from gene introduction to gene expression, and analysis or control of cell differentiation, while minimizing the damage on the cell during the process of introducing a gene or a gene expression-related substance into the cell.

In order to achieve the above object, the inventors have carried out various studies on cell-manipulating techniques for introducing a gene or a gene expression-related substance into a cell and expressing the functions thereof, and developed a novel concept of immobilizing a gene or a gene expression-related substance onto a needle-shaped body, inserting the needle-shaped body into the cell, and holding the gene or the gene expression-related substance in the cell to allow expression of its functions while maintaining the immobilization of the gene or the gene expression-related substance relative to the needle-shaped body.

More specifically, according to a first aspect of the present invention, there is provided a cell-manipulating apparatus comprising a needle-shaped body for immobilizing a gene or a gene expression-related substance thereonto, means for moving the needle-shaped body, means for controlling the position of the needle-shaped body, and means for detecting at least the insertion of the needle-shaped body with respect to a cell.

In the cell-manipulating apparatus according to the first aspect of the present invention, the detecting means may be operable to detect the contact, penetration, insertion and extraction of the needle-shaped body with respect to the cell.

Alternatively, the detecting means may be operable to measure a force acting on the needle-shaped body from the cell. In this case, the detecting means may be a force-variation measuring device incorporated in an atom force microscope or a molecular force probe. The force-variation measuring device may comprise a probe for fixedly mounting the needle-shaped body, a cantilever having the probe at the front edge thereof, and means for detecting the displacement of the cantilever.

In the cell-manipulating apparatus according to the first aspect of the present invention, the needle-shaped body may be a carbon nanotube.

Alternatively, in the cell-manipulating apparatus according to the first aspect of the present invention, the needle-shaped body may be an etched and sharpened silicon probe used in force microscopy.

According to a second aspect of the present invention, there is provided a cell-manipulating method comprising steps of immobilizing a gene or a gene expression-related substance onto a needle-shaped body, inserting the needle-shaped body supporting thereon the gene or the gene expression-related substance into a cell, and holding the gene or the gene expression-related substance in the cell while maintaining the support of the gene or the gene expression-related substance immobilized on the needle-shaped body.

According to a third aspect of the present invention, there is provided a cell-manipulating method comprising steps of immobilizing a gene or a gene expression-related substance onto a needle-shaped body, inserting the needle-shaped body supporting the gene or the gene expression-related substance into a cell, holding the gene or the gene expression-related substance in the cell while maintaining the support of the gene or the gene expression-related substance relative to the needle-shaped body, and extracting the needle-shaped body from the cell while maintaining the support of the gene or the gene expression-related substance immobilized on the needle-shaped body.

### BRIEF DESCRIPTION OF THE DRAWINGS

FIG. 1 is an electron microscopic picture of a probe for fixedly mounting a carbon nanotube, used in one embodiment of the present invention.

FIG. 2 is an optical microscopic picture of Normal Human Epidermal Melanocyte-Neonatal (NHEM-Neo) cells.

FIG. 3 is a schematic diagram of a cell-manipulating apparatus according to one embodiment of the present invention, wherein the reference numeral (1) indicates an atomic force microscope, the reference numeral (2) indicates an incident-light fluorescence microscope, the reference numeral (3) indicates a two-dimensional stage, the reference numeral (4) indicates a cantilever, the reference numeral (5) indicates a probe, the reference numeral (6) indicates a needle-shaped body such as a carbon nanotube attached to the probe, and the reference numeral (7) indicates a cell.

FIG. 4 is a diagram showing a force acting on the cantilever during a operation of introducing the carbon nanotube into the cell. The reference numerals are as described in FIG. 3.

FIG. 5 is an observation picture showing temporal changes of fluorescent protein expression in a melanocyte into which a GFP (green fluorescent protein) gene is introduced in Example 5.

FIG. 6 is an electron microscopic picture of an etched and sharpened silicon probe, used in one embodiment of the present invention.

FIG. 7 is a diagram showing a force acting on the cantilever during an operation of introducing the etched and sharpened silicon probe into a cell.

FIG. 8 is an observation picture showing temporal changes of fluorescent protein expression in a melanocyte into which a GFP gene is introduced in Example 10.

### DETAILED DESCRIPTION OF THE INVENTION

A cell-manipulating apparatus of the present invention comprises a needle-shaped body for immobilizing a gene or a gene expression-related substance thereonto, means for moving the needle-shaped body, means for controlling the position of the needle-shaped body, and means for detecting at least the insertion of the needle-shaped body with respect to a cell.

In the cell-manipulating apparatus of the present invention, the detecting means is preferably operable to detect the contact, penetration, and extraction of the needle-shaped body with respect to the cell, in addition to the insertion. The detecting means may be operable to measure a micro force acting on the needle-shaped body from the cell during the contact, penetration, insertion or extraction of the needle-shaped body with respect to the cell. For example, the detecting means may be a device for measuring a force acting on a probe of an atomic force microscope or a molecular force probe. Specifically, the measuring device may comprise a probe, a cantilever having the probe at the front edge thereof, and means for detecting the displacement of the cantilever, which are incorporated in an atomic force microscope (AFM) or a molecular force probe.

The needle-shaped body may be attached to the probe. Alternatively, the probe itself may be sharpened to a needle-shape. In this case, the apparatus of the present invention utilizes a function of measuring the curve of the force acting on the probe but not a function of measuring an atomic force or molecular force itself. The apparatus may further include means for moving the probe or the cantilever having the probe at the front edge thereof, and means for detecting or controlling the position of the probe. Further, the apparatus is preferably a cantilever-driving type capable of directly handling cultured cells on a plate and accessible from above.

The present invention will be described by taking a case where a probe and associated devices of an AFM are employed therein as an example.

An AFM has a measurement principle of detecting a displacement of a cantilever caused by its bending due to an atomic force to be generated when a micro probe at the front edge of the cantilever is moved close to a sample surface, by means of a laser reflection, and imaging the topography of an sample surface in nanometer order in accordance with the detected data.

In this case, the needle-shaped body to be inserted into a cell with a gene or a gene expression-related substance immobilized thereonto is provided in the probe of the atomic force microscope. The needle-shaped body may be a carbon nanotube, a silicon crystal, a metal crystal such as titanium or zirconium crystal, or a metal oxide such as ZnO. Alternatively, a silicon probe or a silicon nitride probe sharpened to a needle-shape may be used. The needle-shaped body may have a diameter of about 10 nm to 30 μm, preferably 10 to 100 nm.

When the carbon nanotube is used as the needle-shaped body, it may have a desirable diameter of about 10 to 100 nm, which is sufficiently smaller than the size of a cell to allow the cell to be free from its damage otherwise caused during a process of inserting the nanotube into the cell. The silicon probe sharpened to a needle-shape 100 nm in diameter may also be used with low cell damage. The carbon nanotube may have a length of about 0.5 to 200 μm, preferably 2 to 10 μm. The silicon probe may be sharpened to 10 μm in length, sufficient for insertion into a cell.

While there are two types of carbon nanotubes: a single-walled carbon nanotube and a multi-walled carbon nanotube, it is desired to select the multi-walled carbon nanotube because the needle-shaped body of the present invention is required to assure an adequate strength against a lateral bending stress.

For example, when the above carbon nanotube used as the needle-shaped body is attached to the probe, the probe at the front edge of the cantilever is first brought into contact with the front edge of the carbon nanotube fixed to a base, and the contact region is then irradiated with an electronic beam to decomposed a carbon-compound gas by the energy of the electronic beam, under electronic microscope observation. The carbon-compound gas is deposited as amorphous carbon on the contact region to fixedly attach the probe to the carbon nanotube. Then, the carbon nanotube is detached from the base. Alternatively, a cantilever with a carbon nanotube may be formed by growing the carbon nanotube on the surface of the cantilever through a CVD method.

When a silicon probe is sharpened to a needle-shape and used as the needle-shaped body, the silicon probe is etched and is sharpened to a needle shape using a focused ion beam. In the case of silicon nitride probe, such a needle-shaped probe may be formed using the thin hole of a template.

If required in any of the procedures or manipulations described herein, aspiration using a micro-capillary may be used to hold a cell in place during the introduction of the gene or a gene expression-related substance into the cell.

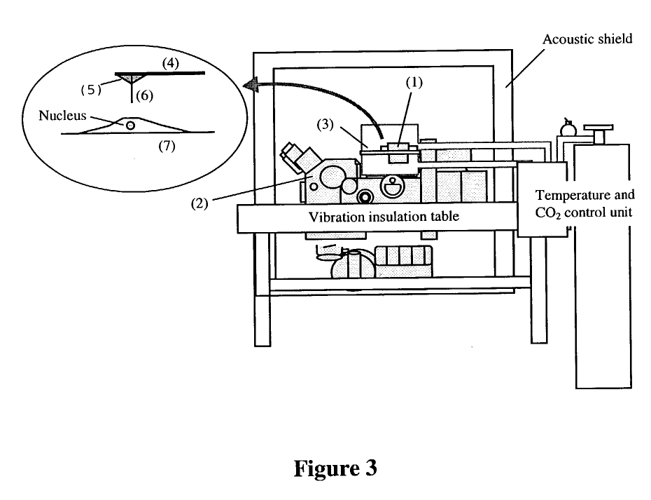

As shown in FIG. 3, a cell-manipulating apparatus according to one embodiment of the present invention comprises an atomic force microscope (1) including a two-dimensional stage (3) for placing a culture vessel thereon, and an incident-light fluorescence microscope (2). The atomic force microscope (1) includes a cantilever (4), a probe (5) fixed to the front edge of the cantilever (4), and a needle-shaped body (6) such as a carbon nanotube attached to the front edge of the probe (5). The probe (5) and the needle-shaped body (6) are disposed above the two-dimensional stage (3). The needle-shaped body (6) is vertically moved by a device (details not shown) for moving the cantilever (4) and a device (details not shown) for controlling the position of the cantilever (4). The cell-manipulating apparatus is operable to detect the bending of the cantilever (4) by a laser reflection and allow a force acting on the needle-shaped body to be monitored.

An operation of manipulating a cell by using the above cell-manipulating apparatus will be described bellow.

A gene or a gene expression-related substance is first immobilized onto the needle-shaped body (6) attached to the probe (5), or a silicon probe sharpened to a needle-shape (6), which is fixed to the front edge of the cantilever (4) (FIG. 3). For example, this immobilization may be achieved by utilizing physical adsorption or avidin-biotin binding using immobilized avidin. When a carbon nanotube is used as the needle-shaped body, the surface of the carbon nanotube itself may be modified in advance through an organic synthesis technique. For example, after modifying the surface of the carbon nanotube so as to expose a functional group such as a carboxyl group to provide a hydrophilic characteristic to the surface, a gene can be covalently-bonded with the functional group through an organic chemical technique using an activating reagent to immobilize the gene onto the surface of the carbon nanotube.

When a silicon or silicon nitride probe sharpened to a needle-shape is used as the needle-shaped body, silane coupling reagents are used to form functional groups on an oxidized silicon or silicon nitride surface. Then, a gene can be covalently-bonded with the functional group through an organic chemical technique using an activating reagent to immobilize the gene onto the surface of the silicon or silicon nitride probe sharpened to a needle-shape.

Then, a culture vessel containing a target cell cultured therein is placed on the two-dimensional stage (3) of the cell-manipulating apparatus, and the two-dimensional stage is moved to a position where the probe (5) of the cantilever is located directly above the target cell in the culture vessel placed on the two-dimensional stage, while monitoring with the incident-light fluorescence microscope (2). The needle-shaped body (6) at the front edge of the probe of the cantilever is gradually moved closer to the cell (7), and finally inserted into the cell (7), while monitoring the displacement of the cantilever (4). After completion of the insertion operation, the needle-shaped body (6) is held at the insertion position to induce gene expression or the like in the immobilized gene on the needle-shaped body. During the insertion operation, the cantilever (4) is displaced due to a repulsive force from the cell when the needle-shaped body is brought into contact with the cell, and then the repulsive force acting on the cantilever (4) is rapidly relaxed to allow the displacement of the cantilever to decrease when the needle-shaped body is penetratingly inserted through the membrane of the cell.

While the repulsing force scarcely decreases as long as the needle-shaped body (6) is held in the cell and the gene expression or the like is induced in the immobilized gene on the needle-shaped body (6), micro displacements will be continuously observed and adequately adjusted. After the gene expression or the like for a given time-period, the needle-shaped body is extracted from the cell (7) to take the gene out of the cell. During the extraction operation, a low frictional force acts on the needle-shaped body from the cell, and the frictional force acts on the cantilever (4) as a low pulling force causing the displacement of the cantilever.

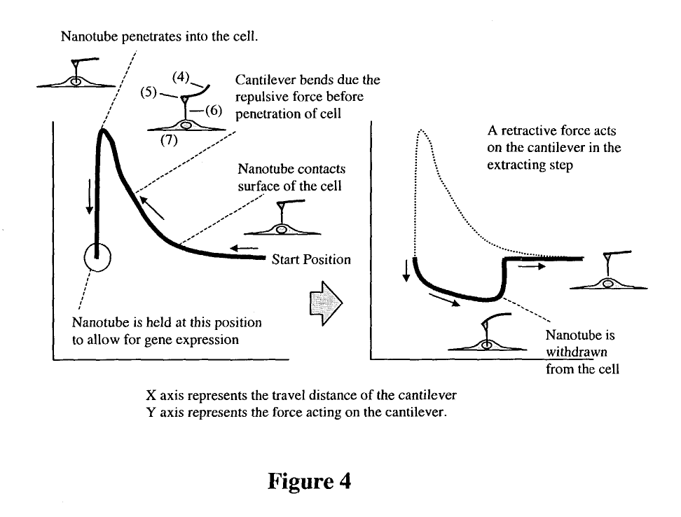

Subsequently, this displacement is restored. This means that the needle-shaped body has been completely extracted from the cell. The above displacements of the cantilever (4) can be monitored on a monitor screen to provide a detail and real-time observation of a series of cell-manipulating operations including the contact between the cell (7) and the needle-shaped body (6), and the insertion, holding in the cell and extraction of the needle-shaped body (6) (see FIG. 4). In addition, the gene expression or another effect caused by the gene or the gene expression-related substance on the needle-shaped body held in the cell can be observed in real time with the incident-light fluorescence microscope of the cell-manipulating device.

As described above, in the present invention a gene or a gene expression-related substance is not permanently held in a cell, and thereby no gene recombinant is formed even during gene expression. Thus, in view of the fact that cell differentiation involves gene expression over a relatively short time-period, the present invention is an optimal method for analyzing the cell differentiation mechanism, or inducing and/or controlling the cell differentiation. In addition, the feature of forming no gene recombinant can advantageously ensure the safety in gene introduction, and potentially eliminates the risks of antigenicity and/or rejection in cell therapy because a genotype identical to that of a patient will be maintained. Thus, the present invention provides one desired technique for gene therapy.

In the present invention, the gene to be immobilized onto the needle-shaped body is not limited to a specific gene. For example, a gene useful for analyzing signal transduction in stem cell generation/differentiation, or causing or potentially causing cell differentiation by its suppressed or stimulated expression in a transcriptional control mechanism may be used. And introduction of a gene related to disease is useful for the pathophysiological analysis. Preferably, the gene comprises at least a promoter and the coding sequence of a polypeptide. Alternatively, mRNA made be used as the gene.

The gene expression-related substance to be immobilized on the needle-shaped body is not limited to a specific substance, but any suitable substance, such as proteins, related to gene responses in cells may be used. For example, it is contemplated that as one of differentiation controls, a repressor protein is introduced to suppress the transcription of genes to be expressed in cells, so as to control the expression of the genes in the genome. Non-limiting examples of other gene-expression-related substances include hormones and organic compounds related to gene expression (i.e., mRNA transcription, protein translation, signal transduction).

The cells used in the present invention are not limited and may include, for example eukaryotic cells such as mammalian cells, insect cells, cells from invertebrate animals, and prokayotic cells such as bacterial cells.

The present invention has been described in conjunction with one specific embodiment primarily comprising the probe, the means for moving the probe, the means for controlling the position of the probe, and the cantilever serving as the detecting means, which are incorporated in the atomic force microscope.

While various Examples of the present invention will be described below, the invention is not limited thereto.

### EXAMPLES

### Example 1

Immobilization of Carbon Nanotube with respect to AFM Probe

A plurality of purified multi-layered carbon nanotubes were subjected to electrophoresis. The nanotubes were moved and turned along the direction of an electric field, and a number of the nanotubes were attracted onto the knife edge of an electrode in such manner that one of the ends of each of the nanotubes was attached to the knife end and the other end was protruded outward. This knife edge with the nanotubes and a cantilever were introduced into a scanning electron microscope (SEM) exclusive to an attaching operation. The SEM includes a three-dimensionally movable stage for bringing the carbon nanotubes into contact with a probe of the cantilever. On this stage, the nanotubes were brought into contact with the probe in a face-to-face relationship, and one of the nanotubes having a length of 3 μm or more allowing the front edge of the nanotube to reach the nucleus of a cell was selected under electron microscope observation.

Under the microscope observation, the selected nanotube and the probe were moved closer to each other, and the movement was stopped when the front edge of the nanotube was brought into contact with the side surface of the probe. Only the contact region was scanned with incident electron beams of the electron microscope for several minutes. Through the scanning, a remaining carbon-compound gas in a vacuum container of the electron microscope was decomposed and deposited as carbon amorphous on the scanned region to fix the nanotube to the probe. During this operation, the nanotube was adjusted to have a tilt angle of 15° with respect to a direction perpendicular to the surface of the cantilever, so that the nanotube extends perpendicularly downward when it is attached to an inclined cantilever holder of the AFM.

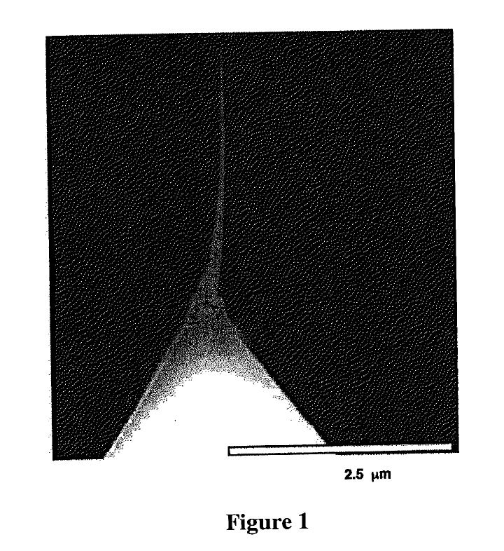

In order to provide mechanical strength to the immobilized region of the nanotube on the probe so as to prevent bending of the nanotube from occurring, the immobilized region was additionally irradiated with the electron beams to deposit amorphous carbon thereon. In this way, an optimal gene-injecting probe having a front-edge diameter of about 10 nm and a length of 3 μm or more could be prepared (see FIG. 1).

### Example 2

Culture of Cells

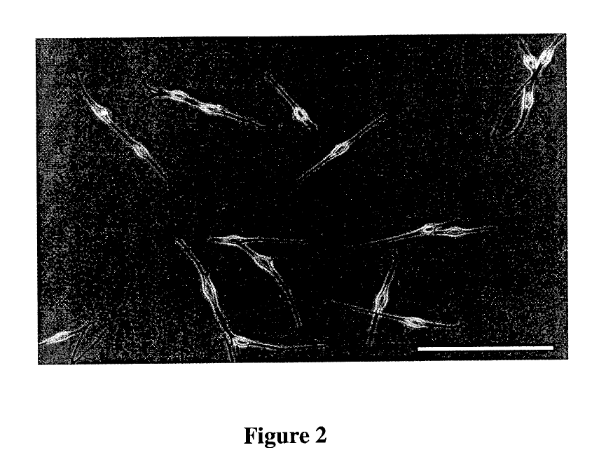

Normal Human Epidermal Melanocyte-Neonatal (NHEM-Neo) cells (Bio Whittaker, Inc., Maryland, USA) were cultured at 37° C. under 5% CO₂ atmosphere using MGM-3 BulletKit (Bio Whittaker, Inc., Maryland, USA) as a culture medium. A 35 mm petri dish was used as a culture vessel. The number 1.6×10⁶ of cells were disseminated on the petri dish, and cultured over night to obtain cells to be used for gene injection (see FIG. 2).

### Example 3

Measurement of Force during Insertion of Carbon Nanotube with Respect to Cell

As shown in FIG. 3, the two-dimensional stage (3) of the atomic force microscope (AFM) (1) was used as a sample stage to facilitate application to a living cell sample, and the culture vessel with the cultured cells (NHEM-Neo) was positioned on the stage. The incident-light fluorescence microscope (2) was integrated with the AFM (1) to allow the sample (7) and the AFM cantilever (4) to be aligned with one another under bright-field observation while illuminating them upward and to allow cell manipulation based on the AFM cantilever to be performed under real-time fluorescent observation using a high-sensitivity CCD (charge-coupled device). The cell-manipulating apparatus was also provided with a chamber capable of controlling temperature and CO₂ to maintain suitable circumstances for cell growing during the cell manipulation using the AFM cantilever (4).

Under the microscope observation, the probe (5) of the cantilever (4) was positioned directly above the center of the target cell on the two-dimensional stage (3). Then, as shown in FIG. 4, the cantilever (4) was gradually moved closer to the cell while monitoring the displacement of the cantilever, and a force acting on the cantilever was observed. During this operation, a specific point where the cantilever was sharply bent by a repulsive force was observed. This result means that the front edge of the nanotube (6) was brought into contact with the cell membrane at the point. When the cantilever was further moved toward the cell, the repulsive force was increased and then sharply relaxed. This result means that the front edge of the nanotube (6) penetrated the cell membrane and entered the inside of the cell. Then, the nanotube (6) was extracted from the cell (7). During the extraction operation, a low pulling force resisting against the extraction of the nanotube was observed, and then relaxed. It can be considered that the nanotube was completely separated from the cell (7) at this re-relaxation point. The contact state between the nanotube (6) and the cell (7) can be intimately observed in real time in accordance with the relationship between the change of the force acting on the cantilever and the distance between the cantilever and the cell observed during the series of operations.

### Example 4

Attachment of Gene with Respect to Carbon Nanotube

The plasmid pQB125 (Wako Pure Chemical Industries, Ltd., Tokyo Japan) comprising a green fluorescent protein (GFP) gene, and the CMV-1E promoter/enhancer exhibiting excellent transcription activity in human cells, was used as the gene to be introduced. 10 μl of a 0.5 μg/ml plasmid solution (10 mM Tris-HCl, pH 8.0, 1.0 mM EDTA) was dropped on the cantilever and attached nanotube probe, and left at room temperature for 30 minutes to achieve immobilization through physical adsorption.

### Example 5

Introduction of Gene by Nanotube

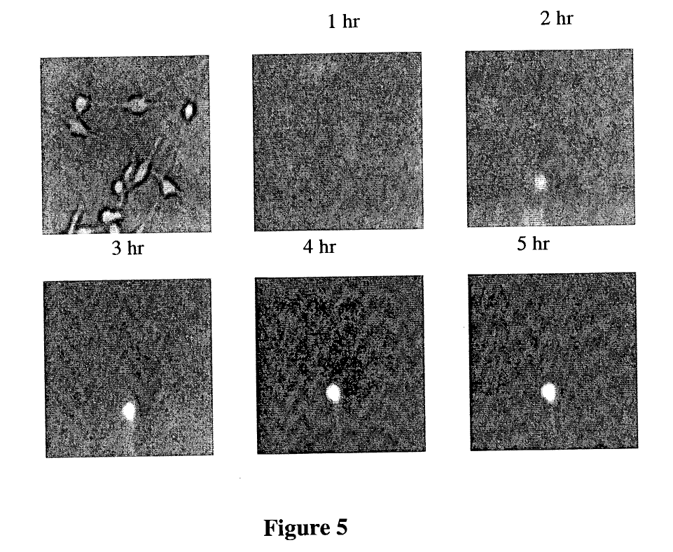

The probe of the cantilever was moved above a position where the nucleus of the cell (NHEM-Neo) can exist or the center region of the cell. Then, the cantilever was gradually moved closer to the cell to insert the nanotube with the immobilized gene into the cell, while observing the displacements of the cantilever. The inserted position was held for 5 hours, and the expression of the gene attached on the nanotube was observed by the fluorescence microscope every 1 hour. As a result, after about 2 hours from the injection of the nanotube, GFP fluorescence was observed only in one cell to which the nanotube was inserted. The strong fluorescence was continuously observed within 4 hours (see FIG. 5). This proved that a gene can be selectively introduced into a single cell.

### Example 6

Etching of Silicon AFM probe

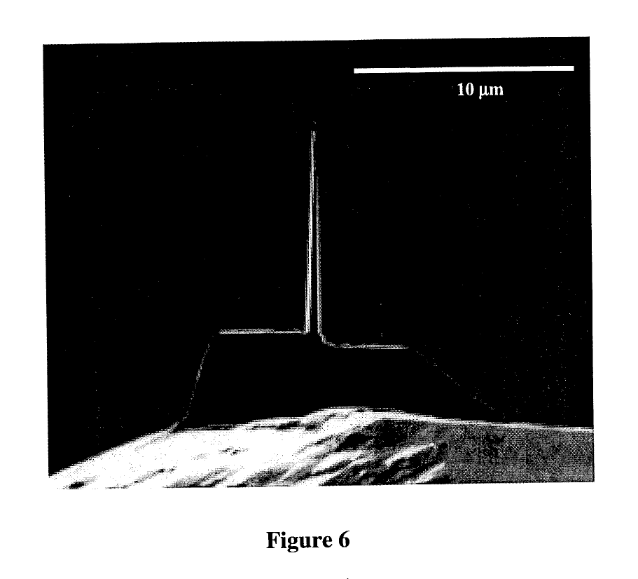

An AFM cantilever probe with a 0.1 N/m spring constant was etched to sharpen the probe to a needle-shape using a focused ion beam. Namely, the etching beam was irradiated from one direction to sharpen the pyramidal probe to a thin shape, then the cantilever was put out, settled in the rotated position of 90 degree, and then the beam was irradiated as above to form the needle-shaped probe. The etched probe is shown in FIG. 6.

### Example 7

Culture of Cells

As in Example 2, Normal Human Epidermal Melanocyte-Neonatal (NHEM-Neo) cells (Bio Whittaker, Inc., Maryland, USA) were cultured at 37° C. under 5% CO₂ atmosphere using MGM-3 BulletKit (Bio Whittaker, Inc., Maryland, USA) as a culture medium. A 35 mm petri dish was used as a culture vessel. The number 1.6×10⁶ of cells were disseminated on the petri dish, and cultured over night to obtain cells to be used for gene injection.

### Example 8

Measurement of Force During Insertion of Silicon Probe Sharpened to Needle-Shape with Respect to Cell

The same apparatus as in Example 3 was used in the operation except that a silicon probe was sharpened to a needle-shape as an alternative for the nanotube. Namely, the two-dimensional stage (3) of the atomic force microscope (AFM) (1) was used as a sample stage to facilitate application to a living cell sample, and the culture vessel with the cultured cells (NHEM-Neo) was positioned on the stage. The incident-light fluorescence microscope (2) was integrated with the AFM (1) to allow the sample (7) and the AFM cantilever (4) to be aligned with one another under bright-field observation while illuminating them upward, and to allow cell manipulation based on the AFM cantilever to be performed under real-time fluorescent observation using a high-sensitivity CCD. The cell-manipulating apparatus was also provided with a chamber capable of controlling temperature and CO₂ to maintain suitable circumstances for cell growth during the cell manipulation using the AFM cantilever (4).

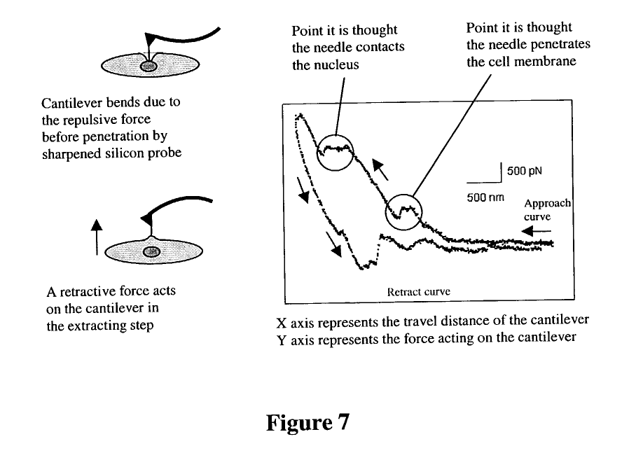

Under the microscope observation, the probe (5) of the cantilever (4) was positioned directly above the center of the target cell on the two-dimensional stage (3). Then, as shown in FIG. 7, the cantilever (4) was gradually moved closer to the cell while monitoring the displacement of the cantilever, and a force acting on the cantilever was observed. During this operation, a specific point where the cantilever was sharply bent by a repulsive force was observed. This result meant that the front edge of the sharpened silicon probe (6) was brought into contact with the cell membrane. When the cantilever was further moved toward the cell, the repulsive force was increased and then sharply relaxed. This result meant that the front edge of the sharpened silicon probe (6) penetrated the cell membrane and entered the inside of the cell. The repulsive force gradually increased again caused by friction between the surface of the sharpened probe and the cell membrane. During continuous insertion, a second force relaxed point thought to be contact with the nucleus membrane was observed.

Then, the sharpened silicon probe (6) was extracted from the cell (7). During the extraction operation, a low pulling force resisting against the extraction of the nanotube was observed, and then relaxed. It can be considered that the nanotube was completely separated from the cell (7) at this re-relaxation point. The contact state between the sharpened silicon probe (6) and the cell (7) can be intimately observed in real time in accordance with the relationship between the change of the force acting on the cantilever and the distance between the cantilever and the cell observed during the series of operations.

### Example 9

Attachment of Gene with Respect to Silicon Probe Sharpened to Needle-Shape

The plasmid pQB125 (Wako Pure Chemical Industries, Ltd., Tokyo Japan) comprising a green fluorescent protein (GFP) gene, and the CMV-1E promoter/enhancer exhibiting excellent transcription activity in human cells, was selected and a DNA fragment containing the promoter and the GFP structural gene was amplified by the polymerase chain reaction (PCR) method. The primer used in the PCR can be bound to avidin because the primer was biotinylated. The surface of the sharpened silicon probe was oxidized using an ozone cleaner. Then, avidin was immobilized on to the sharpened silicon probe using a silane coupling reagent and a bifunctional coupling reagent. The solution of the biotinylated GFP DNA fragment was dropped on the cantilever and the sharpened silicon probe to immobilize the biotinylated DNA fragment on the avidin-coated probe.

### Example 10

Introduction of Gene by Silicon Probe Sharpened to Needle-Shape

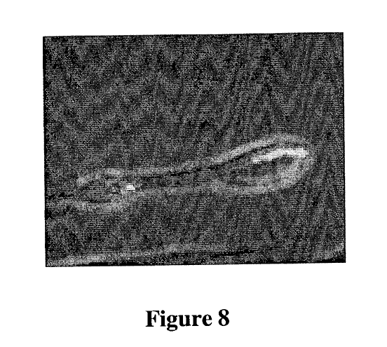

The probe of the cantilever was moved above a position where the nucleus of the cell (NHEM-Neo) can exist or the center region of the cell. Then, the cantilever was gradually moved closer to the cell to insert the sharpened silicon probe with the immobilized gene into the cell, while observing the displacement of the cantilever. The insert position was held for 5 hours, and the expression of the gene attached to the sharpened silicon probe was observed by the fluorescence microscope every 1 hour. As a result, after about 2 hours from the injection of the sharpened silicon probe, GFP fluorescence was observed only in one cell to which the sharpened silicon probe was inserted. The strong fluorescence was continuously observed over 4 hours (see FIG. 8). FIG. 8 shows the cell after 2 hours. This proved that a gene can be selectively introduced into a single cell and successfully expressed therein.

As mentioned above, according to the present invention, the cell damage can be minimized during the process of introducing a gene or a gene expression-related substance into a cell, and the expression of the gene or the effect from the gene-related substance can be observed in real time. In addition, the gene or the gene expression-related substance is not permanently held in the cell, and thereby no gene recombination will occur even if there is gene expression. Thus, in view of the fact that cell differentiation involves gene expression over a relatively short time-period, the present invention is an optimal method for analyzing the cell differentiation mechanism, or inducing and/or controlling cell differentiation. The present invention provides a desired technique applicable to gene therapy and/or cell therapy, particularly for cancer or Alzheimer's disease.

Moreover, the present invention may be used in a drug-effectiveness test in conjunction with tailor-made medicines. For example, a marker gene of inhibition of cancer growth may be immobilized on a needle-shaped body and introduced into a cell or tissue from a cancer patient during dosage with a test drug. If the marker gene is expressed in the cell or tissue upon treatment with the test drug, it may indicate that the drug will be effective in the patient.

The skilled artisan will readily envision other uses for the present invention. Additional examples for use include the fields of apoptosis studies, cellular differentiation studies, gene therapy studies, pharmacological studies, aging studies and so on. In each case, the present invention would provide analysis with high time resolution.

What is claimed is:

1. A cell-manipulating apparatus comprising:
a needle-shaped body for immobilizing a gene or a gene expression-related substance thereonto;
means for moving said needle-shaped body;
means for controlling the position of said needle-shaped body; and
means for detecting at least the insertion of said needle-shaped body with respect to a cell.

2. The cell-manipulating apparatus as defined in claim 1, wherein said detecting means is operable to detect one or more of contact, penetration, insertion and extraction of said needle-shaped body with respect to said cell.

3. The cell-manipulating apparatus as defined in claim 1 or 2, wherein said detecting means is operable to measure a force acting on said needle-shaped body from said cell.

4. The cell-manipulating apparatus as defined in claim 3, wherein said detecting means is a force-variation measuring device incorporated into an atom force microscope or a molecular force probe.

5. The cell-manipulating apparatus as defined in claim 4, wherein said force-variation measuring device comprises a probe for fixedly mounting said needle-shaped body, a cantilever having said probe at the front edge thereof, and means for detecting the displacement of said cantilever.

6. The cell-manipulating apparatus as defined in claim 4, wherein said force-variation measuring device comprises a probe itself sharpened into a needle-shaped body, a cantilever having said probe at the front edge thereof, and means for detecting the displacement of said cantilever.

7. The cell-manipulating apparatus as defined in claim 1, wherein said needle-shaped body is a carbon nanotube.

8. A cell-manipulating method comprising:
immobilizing a gene or a gene expression-related substance onto a needle-shaped body;
inserting said needle-shaped body supporting thereon the gene or the gene expression-related substance into a cell; and
holding the gene or the gene expression-related substance in said cell while maintaining the support of the gene or the gene expression-related substance relative to said needle-shaped body.

9. A cell-manipulating method comprising:
immobilizing a gene or a gene expression-related substance onto a needle-shaped body;
inserting said needle-shaped body supporting thereon the gene or the gene expression-related substance into a cell;
holding the gene or the gene expression-related substance in said cell while maintaining the support of the gene or the gene expression-related substance relative to said needle-shaped body; and
extracting said needle-shaped body from said cell while maintaining the support of the gene or the gene expression-related substance relative to said needle-shaped body.
